(19) United States
(12) Patent Application Publication
(10) Pub. No.: US 2011/0184524 A1
(43) Pub. Date: Jul. 28, 2011

(54) IMPLANT FOR INSERTION BETWEEN VERTEBRAL BODIES OF THE SPINAL COLUMN

(76) Inventors: Nelli Wiedenbeck, Ulm (DE); Stefan Breuer, Erbach (DE); Sven Hamich, Ulm (DE)

(21) Appl. No.: 12/990,961

(22) PCT Filed: Sep. 19, 2008
(86) PCT No.: PCT/DE2008/001563

§ 371 Date: Dec. 9, 2010

(51) Int. Cl. A61F 2/44

(52) U.S. Cl. 623/17.16

(57) ABSTRACT

An implant (1) for insertion between vertebral bodies of the spinal column as a spacer for intervertebral disks removed from the spinal column, vertebrae, or vertebrae portions, comprises a first implant part (2) and a second implant part (3), which can be displaced relative to each other in the direction of the coaxial longitudinal axes thereof for changing the length of the implant (1), and further comprises an attachment plate (5a, 5b), which can be connected at the free end of at least one of the two implant parts (2, 3) in an orientation that is substantially perpendicular to the longitudinal axis using means for detachable fastening. The attachment plate (5a, 5b) has a basic circular shape (8), wherein the basic circular shape (8) is modified at the circumference by superpositions of substantially circular plate extensions (9, 10) having different diameters and/or different degrees of overlap with the basic circular shape (8).

The invention relates to an implant for insertion between vertebrae of the spinal column as a replacement for intervertebral disks, vertebrae, or vertebral parts removed from the spinal column, having a first and a second part that are adjustable relative to one another in the direction of their coaxial longitudinal axes to change the length of the implant.

Such an implant known, for example, from DE 103 11 477 [US 20004/0210312] has proven itself in practice and is characterized by its excellent promotion of the formation of an osseous connection between the vertebrae, with individual adaptability of the implant to the conditions present in situ, which adaptability is achieved in that the first and second parts are adjustable in the direction of their coaxial longitudinal axes, and an attachment plate can be releasably attached to at least one free end.

In their efforts to further develop this proven implant for use under extreme stress conditions of the spinal column, the applicants have submitted the interactions between this attachment plate of the implant and a bottom face of a vertebra or a top face of a vertebra, respectively, to thorough investigations, during which it was possible to determine special dependencies of the ability of the bottom face of a vertebra or the top face of a vertebra to withstand stress.

The object of the invention is therefore to configure an implant of the type described above in such a manner that when using the implant for extreme stress conditions, improved protection of the vertebrae can be achieved.

This object is attained with an implant as described above in that the attachment plate has a circular body and that the circular body is formed, on its circumference, by the provision of essentially circular plate extensions having different diameters and/or different degrees of overlap with the circular body. The advantage that better load distribution, in situ, among regions of the bottom faces or the top faces of the vertebrae that are particularly well able to withstand stress is achieved with the provision of the circular plate extensions, by means of the circular configuration, without unnecessarily increasing the degree of coverage above the attachment plate and thereby preventing bone material from growing into the implant. The edge regions of the bottom faces or the top faces of the vertebrae, in particular, have increased strength as compared with the inner regions of the bottom faces or top faces of the vertebrae, so that greater load absorption at the edge regions is gentler on the vertebrae.

For these reasons, it is particularly advantageous if the circular plate extensions comprise a part that forms a widened neck section relative to the circular body. In this way, better load absorption at the edge regions is made possible.

In order to allow further adaptation of the implant to the shape of the bottom face or of the top face of the vertebra and gentler placement of stress on the bottom face or the top face of the vertebra, the circular plate extensions having the widened neck section can narrow in height from the widened neck section to the outer edge, so that a wedge-shaped cross-section can be achieved.

Preferably, in the implant, the circular body has an inner ring, and the essentially circular plate extensions form an outer, correspondingly shaped ring. An advantage of this embodiment lies in that the hole through the center of the attachment plate, in particular, promotes the formation of an osseous connection between the vertebrae.

It has proven to be particularly advantageous if the circular plate extensions are provided along the outer edge of the attachment plate at least partly according to increasing size or decreasing size. This formation of the attachment plate allows suitable distribution of the pressure surface without having to increase the area of the attachment plate, so that the desired promotion of the formation of an osseous connection is retained for this implant.

In an advantageous embodiment of the implant, the attachment plate has an outwardly convex edge region extending over an angle of 80° to 120° at its outer edge. Alternatively and/or additionally, the attachment plate can have an outwardly concave edge region extending over an angle of 5° to 40° at its outer edge. It is furthermore practical, for the implant, if the attachment plate is shaped to have mirror symmetry, with an axis of symmetry from the center of the concave edge region to the center of the convex edge region.

It is particularly useful for the load behavior if the circular plate extensions are of increasing size from the convex edge region to the concave edge region.

Furthermore, the present invention relates to an attachment plate for an implant according to one of the preceding aspects.

In the following, the invention will be explained in greater detail with reference to an illustrated embodiment shown in the drawing. Therein:

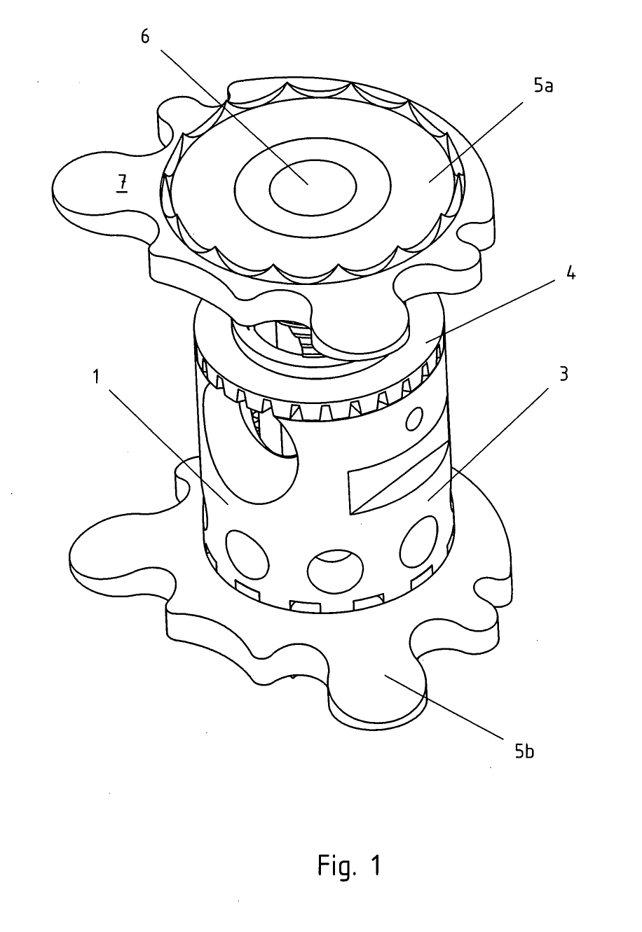

FIG. 1 is a perspective view of an implant consisting of a first implant part and a second implant part and having an attachment plate according to the present invention,

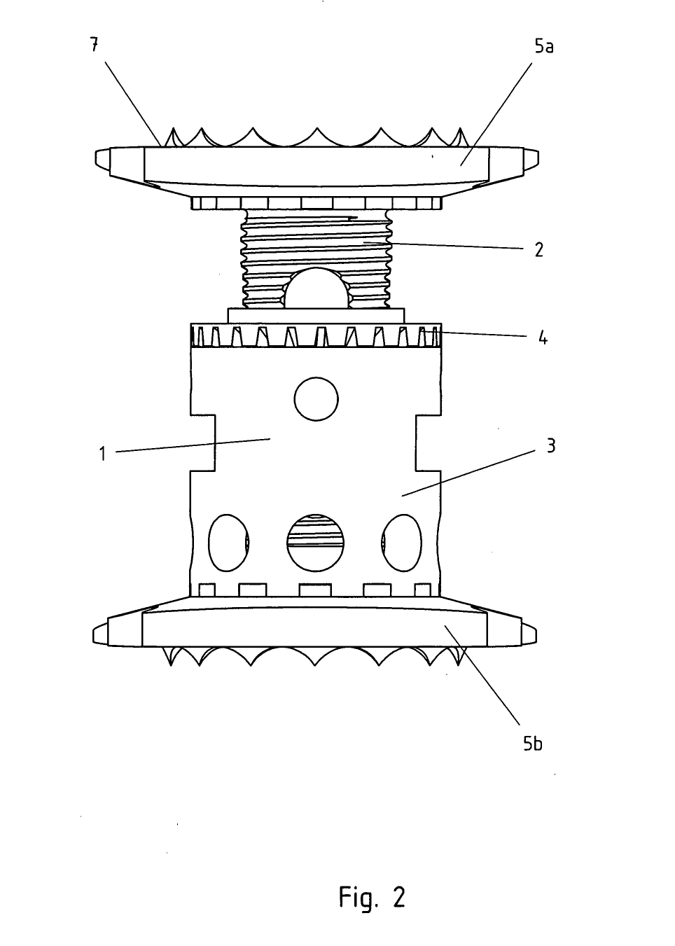

FIG. 2 is a side view of the implant of FIG. 1;

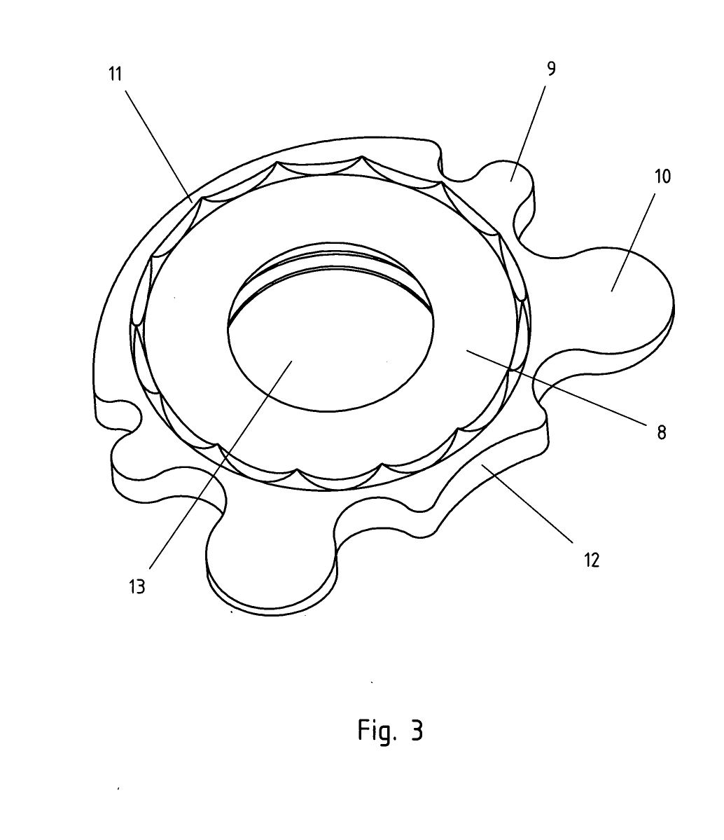

FIG. 3 is a perspective detail view of a top of an upper attachment plate according to the present invention, and

FIG. 4 is a perspective detail view of a bottom side of an upper attachment plate according to the present invention.

In FIGS. 1 and 2, an implant 1 is shown that serves for insertion between vertebrae of the spinal column not shown in the drawing as a replacement for intervertebral disks, vertebrae, or vertebra parts removed from the spinal column. The implant 1 comprises a first implant part 2 and a second implant part 3 that are adjustable relative to each other in the direction of their coaxial longitudinal axes to change the length of the implant 1. The change in length can be carried out by way of a rotatable threaded ring 4 carried on the second implant part 3, for example.

The implant 1 furthermore has at least one attachment plate, in the embodiment shown an upper attachment plate 5a and a lower attachment plate 5b that are secured by releasable attachment means to the outer ends of the two implant parts 2, 3, in an orientation that is essentially perpendicular to the longitudinal axis of the implant 1, and the upper attachment plate 5a and the lower attachment plate 5b can be shaped differently, to fit the shape of the lower and upper faces of the flanking vertebrae.

FIGS. 3 and 4 show in detail a perspective view of a top side and a bottom side of an upper attachment plate 5a according to the present invention. The upper attachment plate 5a has a circular body 8 whose outer edge is formed with generally circular first and second plate extensions 9 and 10. The second plate extension 10 has a larger diameter than the first plate extension 9, and the first plate extension 9 and the second plate extension 10 can differ in their extent of projection from the circular body of the upper attachment plate 5a.

In the implant 1, the circular body 8 can comprise an inner ring having a hole 13. The projecting first and second plate extensions 9 and 10 form an outer, correspondingly shaped ring, in which outer parts of the second plate extensions 10 project beyond the outer ring. Furthermore, each second plate extension 10 can have a widened neck section and can narrow in height from the widened neck section to its outer edge, so that a wedge-shaped cross-section is formed that projects beyond the outer ring at its tip.

At the outer edge, the upper attachment plate has an outwardly convex edge region 11 over an angle of 80° to 120° at its edge. Opposite the convex edge region, an outwardly concave edge region 12 extends over an angle of 5° to 40°. Preferably, the upper attachment plate 5a is shaped to have mirror symmetry, with an axis of symmetry from the center of the concave edge region 12 to the center of the convex edge region 11. In particular, the first and the second plate extensions 9, 10 can be of increasing size from the convex edge region 11 to the concave edge region 12.

### REFERENCE SYMBOL LIST

1 implant

2 first implant part

3 second implant part

4 rotatable threaded ring

5a upper attachment plate

5b lower attachment plate

6 hole in the attachment plate

7 face that faces the vertebra

8 circular body of the attachment plate

9 first circular plate extensions

10 second circular plate extensions with widened necks

11 convex edge region

12 concave edge region

13 hole

1. An implant for insertion between vertebrae of the spinal column as a replacement for intervertebral disks, vertebrae, or vertebra parts removed from the spinal column, having
a first implant part and a second implant part that are adjustable relative to one another in the direction of their coaxial longitudinal axes to change the length of the implant, and
an attachment plate that can be secured by releasable attachment means to the free end of at least one of the two implant parts in an orientation essentially perpendicular to the longitudinal axis, the attachment plate having a circular body and that the circular body is formed, on its circumference, by the provision of essentially circular plate extensions having different diameters and/or different degrees of overlap with the circular body.

2. The implant according to claim 1, wherein the circular plate extensions each comprise a part forming a widened neck section relative to the circular body.

3. The implant according to claim 2, wherein at least a part of the circular plate extensions narrows in height from the widened neck section to the respective outer end.

4. The implant according to claim 1 wherein the circular plate extensions are provided, at least in part, according to increasing size or decreasing size, along the outer edge of the attachment plate.

5. The implant according to claim 1 wherein the circular body comprises an inner ring having a hole, and that the essentially circular plate extensions form an outer, correspondingly shaped ring.

6. The implant according to claim 1 wherein the attachment plate has at its outer edge an outwardly convex edge region extending over an angle of 80° to 120°.

7. The implant according to claim 6 wherein the attachment plate has at its outer edge an outwardly concave edge region extending over an angle of 5° to 40°.

8. The implant according to claim 7, wherein the circular plate extensions are provided at the outer edge with increasing size, from the convex edge region to the concave edge region.

9. The implant according to claim 7, wherein the attachment plate is shaped with mirror symmetry, with a plane of symmetry from the center of the concave edge region to the convex edge region.

10. (canceled)
